(19) United States
(12) Patent Application Publication
(10) Pub. No.: US 2016/0374593 A1
(43) Pub. Date: Dec. 29, 2016

(54) SKINFOLD CALIPER

(71) Applicant: Robert Martin, Glendale, AZ (US)

(72) Inventor: Robert Martin, Glendale, AZ (US)

(21) Appl. No.: 14/748,804

(22) Filed: Jun. 24, 2015

(51) Int. Cl. A61B 5/107; A61B 5/00

(52) U.S. Cl. CPC A61B 5/1075; A61B 5/4872; A61B 5/0053

(57) ABSTRACT

A skinfold caliper for self-measuring a skinfold thickness to calculate body fat, comprising, a first hand and a second hand and an integrated pressure sensor for sensing the pressure applied to a skinfold when the skin contact tips of the caliper hands are engaged with the base of a skinfold held by an opposing hand. When the optimum pressure is achieved, a button with a raised dome attached to the sensor emits an audible click to signal to the user, to stop applying further pressure. The skinfold caliper has a measurement ruler having a metric scale with measurements in millimeters on both sides of the ruler so that the caliper can be read from both sides by a right handed as well as a left handed person. A double-sided marker that slides along the ruler and indicates results of a skinfold measurement can be removed and reinstalled on the ruler.

### FIELD OF THE INVENTION

The present invention relates generally to instruments for measuring body fat. More particularly, the invention relates to a skinfold caliper for self-measuring skinfold thickness to calculate body fat from those skinfold thickness measurements.

### BACKGROUND OF THE INVENTION

Body fat is a term that is anathema to a person who is fixated on their body weight and appearance. It is also a term that has negative connotations to individuals who are overweight and ungainly in appearance due to their excess weight and who may also suffer from illnesses associated with their excess weight. Therefore, the primary aim of any exercise and/or dietary regimen to reduce body weight, necessarily involves, reducing the total percentage of body fat, which in turn requires determining body fat by measuring body fat.

Determining body fat requires measuring the skinfold thickness on prescribed locations on the body, such as the upper arm, abdomen, thigh and hip, typically on the right side of the body. This process involves, grasping a fold of skin and the subcutaneous fat directly underneath the skin on these body locations by hand, pulling the skin and the subcutaneous fat away from the underlying muscle tissue, and by means of a skinfold thickness measuring instrument, taking the measurements of the skinfold thickness. The skinfold thickness measurements are then input into one of several formulas to calculate the body fat percentage of an individual. Measuring the skinfold thickness to determine body fat percentages has always been a challenging proposition over the ages. In the past, the determination of body fat using the skinfold thickness measurement method involved the use of bulky equipment and the assistance and dependence on personnel who were trained in the use of such equipment.

In modern times, skinfold calipers have emerged as a simpler and more acceptable means to measure skinfold thickness to calculate body fat, primarily because of their portability, convenience and ease of use. To execute a skinfold thickness measurement using calipers, a user would typically pick up a layer of skin and the underlying subcutaneous fat with their thumb and forefinger on one hand, either right or left hand, and holding the caliper in the opposing hand, gently close the caliper tips around the base of the skinfold thickness while the user's thumb and forefinger on the hand holding the skinfold, maintain the skinfold hold until the skinfold thickness measurement is completed by the caliper. Measuring the skinfold thickness on prescribed locations of the body using skinfold calipers and calculating the body fat from these measurements using body density formulas commonly used in the industry, such as Jackson-Pollock 3, Jackson-Pollock 7 and Dumin-Womersley, provides a fairly accurate measurement of the total body fat and the percentage of body fat, versus lean body mass of an individual.

Although the use of skinfold calipers to measure skinfold thickness to calculate body fat have in great part replaced the bulky and complex body fat measurement equipment of the past, the majority of the calipers in use today require the assistance of at least one other individual to accurately read the skinfold thickness measurements indicated on the calipers at the time the measurement is taken. The skinfold caliper of the present invention is, a self-measuring tool that does not require the assistance of another individual to read the skinfold thickness measurements using the caliper. U.S. Pat. No. 5,156,161 to Lollar and U.S. Pat. No. 4,127,112 to Sherlock et al., describe skinfold calipers having substantially similar features. The calipers in these prior art require the use of a pressure sensitive cantilever arm engaging with one of the main arms of the caliper to apply pressure to take skinfold measurements. Additionally, the measurement rulers on these prior art calipers have the measurement readings only on one side of the ruler so that, the person using the caliper can use it only with the measurement ruler facing up.

The present invention overcomes these short comings in the prior art by providing a skinfold caliper which does not require the use of a pressure sensitive cantilever arm to take accurate skinfold thickness measurements and has a measurement ruler with measuring scales in millimeters (mm) on both sides of the ruler so that the caliper can be held with either side facing the user to enable either a right handed or left handed person to use the caliper to take skinfold thickness measurements. These and other exemplary features of the skinfold caliper of the present invention will become apparent and obvious to one skilled in the art through the summary of the invention, detailed description of the invention through the drawings, and the appended claims.

### SUMMARY OF THE INVENTION

The present invention is a lightweight and portable skinfold caliper for measuring skinfold thickness to calculate body fat without the assistance of a professional or another individual. The ergonomic design, structure and features of the skinfold caliper of the invention is an improvement over prior art self-measurement calipers.

The exemplary embodiment of the present invention is comprised of a first arm and a second arm seamlessly connected at their lower ends without any hinges, screws or other means to form one structure and allowing pivotal movement of both arms with respect to each other in either an open or closed position of the arms. In this embodiment of the invention, one arm of the caliper has a pressure sensor integrated within a thumb grip button situated halfway between the lower end of the arm and the tip of the arm. The thumb grip button has an embedded raised metal dome that is approximately 12 mm wide to provide a slight tactile feel and produces an audible click sound when the optimal skinfold compression is achieved, signaling the user to discontinue applying pressure to the skinfold with the caliper jaws situated at the top ends of each arm. In this embodiment of the invention, the second arm of the caliper has an indentation midway on the arm to engage either the middle finger, index finger, or the ring finger to exert pressure on the second caliper arm when the caliper is in use. When the caliper is thus held between the thumb and either the middle finger, index finger, or the ring finger, the two arms of the caliper can be compressed to bring both arms closer together so that the tips of the caliper arms can pinch the base of the skinfold while the user's opposing hand maintains the hold on the skinfold when taking the skinfold thickness measurement using the caliper.

In the exemplary embodiment of the skinfold caliper of the present invention, the two arms of the caliper hold a measurement ruler between them. In this embodiment of the skinfold caliper of the invention, the measurement ruler has measurements in millimeters (mm) on both sides of the ruler. The measurement ruler further has a removable ruler marker which travels the length of the measurement ruler to register the final, post-measurement thickness of the skinfold.

Although, the present invention has thus been described with reference to its exemplary embodiment, this embodiment should not be construed as a limitation on the scope of the invention. It is to be understood by those skilled in the art, that the invention can be implemented in embodiments other than the one described in this summary of the invention. The advantages and features of the exemplary embodiment of the skinfold caliper of the present invention as thus described in this summary of the invention will be better understood through the detailed description of the invention with the accompanied drawings and the claims that follow.

### BRIEF DESCRIPTION OF THE DRAWINGS

FIG. 1 is a perspective view of the exemplary embodiment of the skinfold caliper of the present invention.

FIG. 2 is another perspective view of the skinfold caliper of the invention.

FIG. 3 is yet another perspective view of the skinfold caliper of the present invention.

FIG. 4 is an exploded view of the parts of the skinfold caliper of the present invention.

FIG. 5 is a vertical view of the skinfold caliper of the present invention.

FIG. 6 is a side view of the skinfold caliper of the present invention.

FIG. 7 is a top elevation view of the skinfold caliper of the present invention.

### DETAILED DESCRIPTION OF THE INVENTION

The present invention is a skinfold caliper for self-measurement of skinfold thickness to evaluate body fat. The caliper of the present invention has an ergonomic design with a measuring ruler having a metric scale calibrated in millimeters on both sides of the ruler so that measurements can be read from either side of the ruler making it possible for both right handed and left handed individuals to take self-measurements of their skinfold thickness. Additionally, the caliper of the present invention uses an integrated pressure sensor with a button that produces an audible sound to alert the user to stop applying pressure after the ideal skinfold compression is achieved.

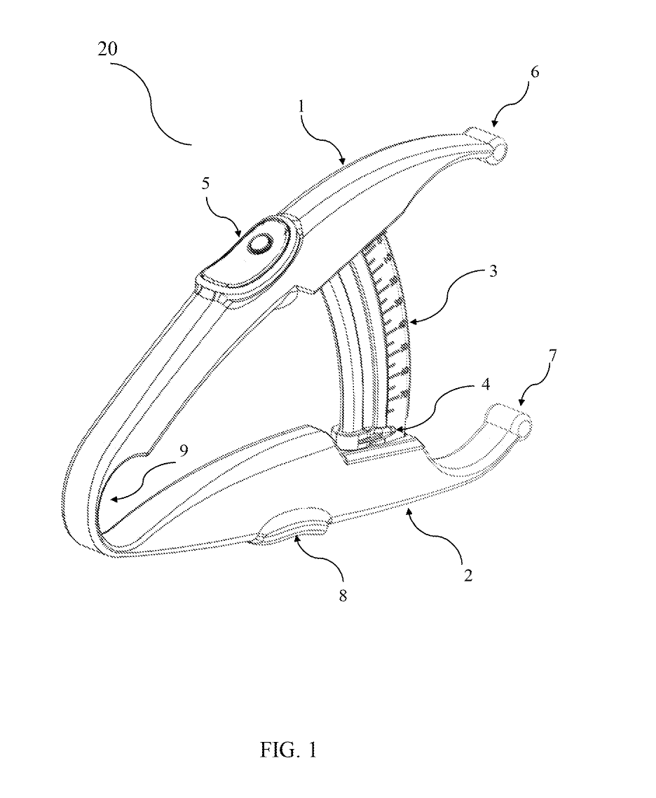

Referring now to the drawings wherein like numerals represent like components in the several views presented and discussed, and more particularly referring now to FIG. 1 the figure is a perspective view of the exemplary embodiment of the skinfold caliper 20 of the present invention illustrating the exemplary features of the caliper. The main body of the caliper comprises a first arm 1 and a second arm 2 with a first jaw 6 at the tip of the first arm 1 and a second jaw 7 at the tip of the second arm 2. The body of the caliper is molded from plastic achieved through a single molding process resulting in a seamless engagement means 9 connecting the lower ends of the first arm 1 and the second arm 2 without the use of screws, hinges, or other connecting means. The caliper 20 has an integrated spring-like shape which allows it to automatically fully open upon the completion of a skinfold thickness measurement. The jaws 6 and 7 at the tips of the first arm 1 and the second arm 2 commonly referred to as skin contact points are rounded and therefore, gentler on the skin, causing less discomfort when pressure is applied to the skinfold to take the measurements, compared to prior art caliper tips which are generally flat shaped and often not as comfortable when touching the skinfold.

Referring again to FIG. 1 the figure also shows a perspective view of a measurement ruler 3 held between the first arm 1 and the second arm 2. In the exemplary embodiment of the caliper, the ruler 3 would have measurements in millimeters (mm) marked on both sides of the ruler 3. This facilitates reading measurements from either side of ruler 3 making it possible for both right handed and left handed individuals to easily and comfortably take their skinfold thickness measurements using the skinfold caliper 20 of the invention. In this embodiment of the skinfold caliper 20 the measurement ruler 3 has a double-sided ruler marker 4 that is capable of sliding between the two ends of the ruler 3. In this embodiment, the ruler marker 4 is removable and re-attachable on the inside part of the ruler 3, providing additional flexibility in the use of the caliper 20 when taking skinfold thickness measurements.

Referring again to FIG. 1 the first arm 1 has an integrated pressure sensor embedded inside the thumb grip button 5 and has a raised dome shape to provide a slight tactile feel and produces an audible click sound when the optimal skinfold compression is achieved. When taking the skinfold thickness measurement, the button 5 on the first aim 1 is pressed down with the thumb so that the first arm 1 can exert the required pressure to the skinfold and when the optimal skinfold compression is achieved, an audible click sound is emitted by button 5 to signal to the user to discontinue applying pressure to the skinfold with the caliper jaws 6 and 7. Once the pressure is released, the button 5 returns to its original position with the help of an integrated leaf spring incorporated into the underside of button 5. The caliper 20 further has an indentation 8 midway through the length of the second arm 2 to provide grip for either the middle finger, index finger, or the ring finger when exerting pressure on the second arm 2.

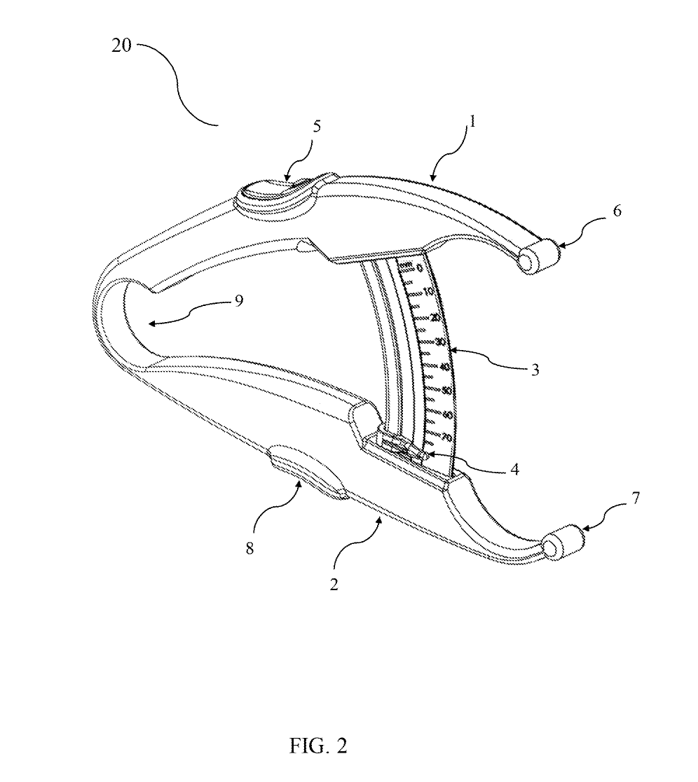

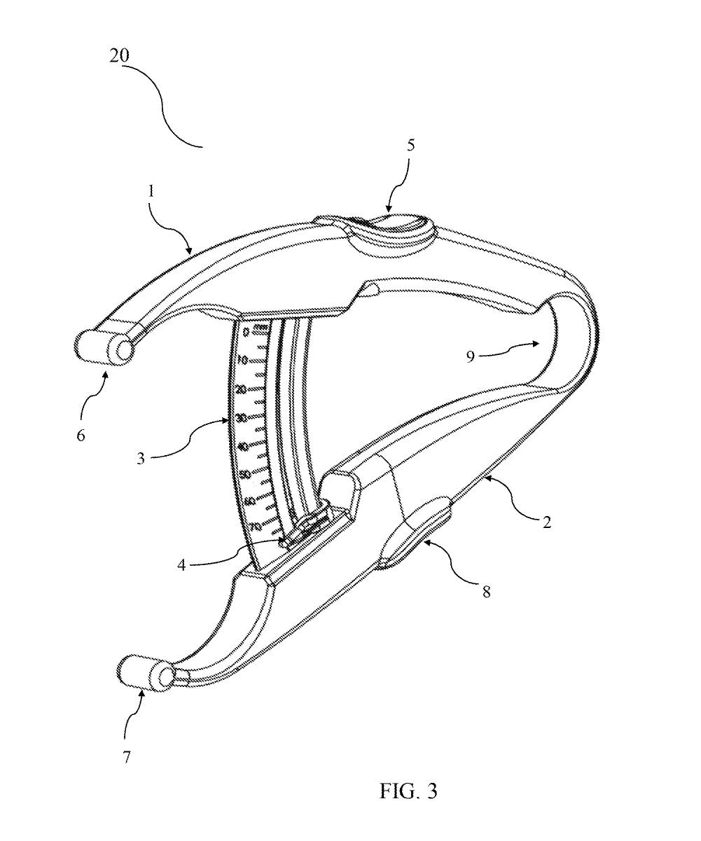

FIG. 2 and FIG. 3 are two perspective views of the skinfold caliper 20 of the present invention with a more prominent view of the rounded jaws 6 and 7 which are the skin contact points for pinching the base of the skinfold held by the opposing hand. Having the jaws 6 and 7 thus rounded makes the tips gentler on the skin allowing for a more comfortable experience when the jaws 6 and 7 are placed on opposing sides of a skinfold on a prescribed part of the body, and compressed with the first arm 1 and the second arm 2. Once the first arm 1 is closed over the skinfold through jaw 6 and the pressure from the thumb exerted on the button 5 and the second arm 2 is likewise closed upward and inward with the opposing finger which could be either the middle finger, index finger, or the ring finger placed on the indentation 8 on the second arm 2 with jaw 7 pinching the skinfold, the ruler marker 4 would slide along the ruler 3 and register a measurement which can then be read and noted. To reset the ruler marker 4 the user would manually slide the ruler marker 4 back to its starting position at the far end of ruler 3. Once the optimal pressure is reached, an integrated pressure sensor within the button 5 signals by emitting an audible, “click” sound to indicate to the user that the optimal skinfold compression has been achieved and to discontinue applying further pressure to the skinfold through jaws 6 and 7. Once the pressure is released from the first arm 1 and second arm 2 the jaws 6 and 7 are released from the skinfold and they along with the two arms of the caliper return to their original positions. The button 5 also returns to its original position with the help of an integrated leaf spring incorporated into the underside of button 5.

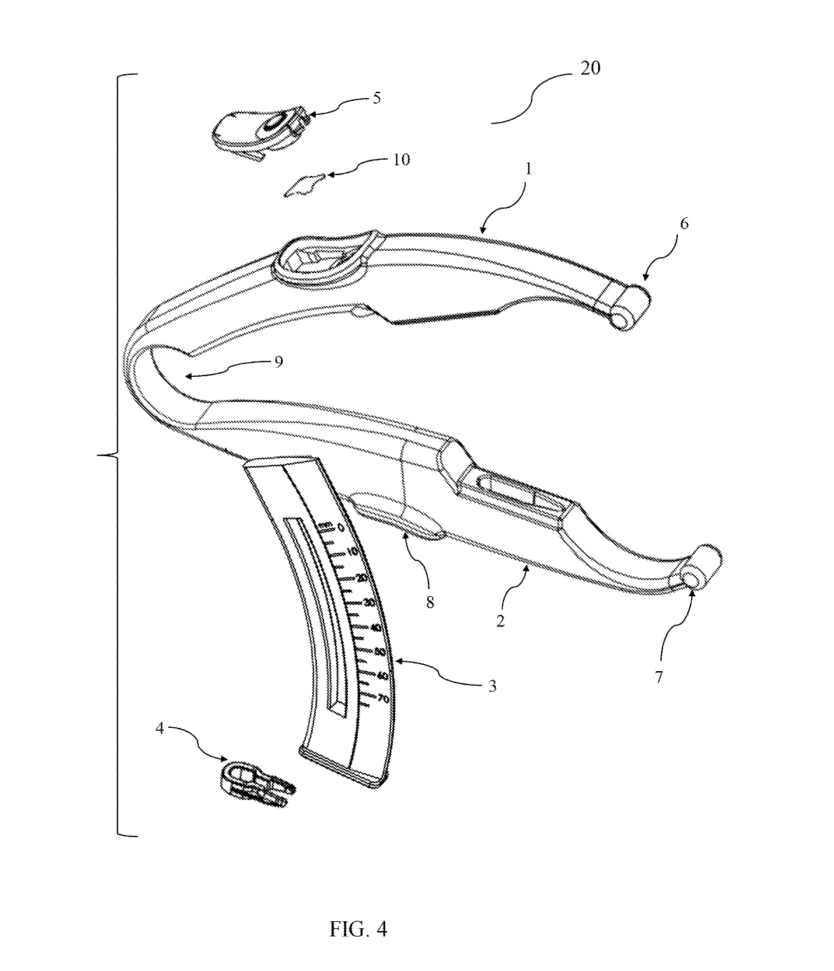

FIG. 4 is an exploded view of the parts of the skinfold caliper of the present invention. The measurement ruler 3 as described previously is shown detached from the main body of the skinfold caliper 20. The ruler 3 has a scale marked in millimeters on both sides of ruler 3 enabling the reading of the skinfold thickness measurements from both sides of ruler 3. The ruler marker 4 is double-sided and detachable from ruler 3, and can be inserted back on ruler 3. The ruler marker 4 slides along ruler 3 and indicates results of a skinfold thickness measurement. In this exploded view of the skinfold caliper 20 the detached button 5 reveals the metal dome 10 that provides a slight tactile feel and an audible click sound when the optimal skinfold compression is achieved and signals the user to discontinue applying pressure to the skinfold with the caliper jaws 6 and 7.

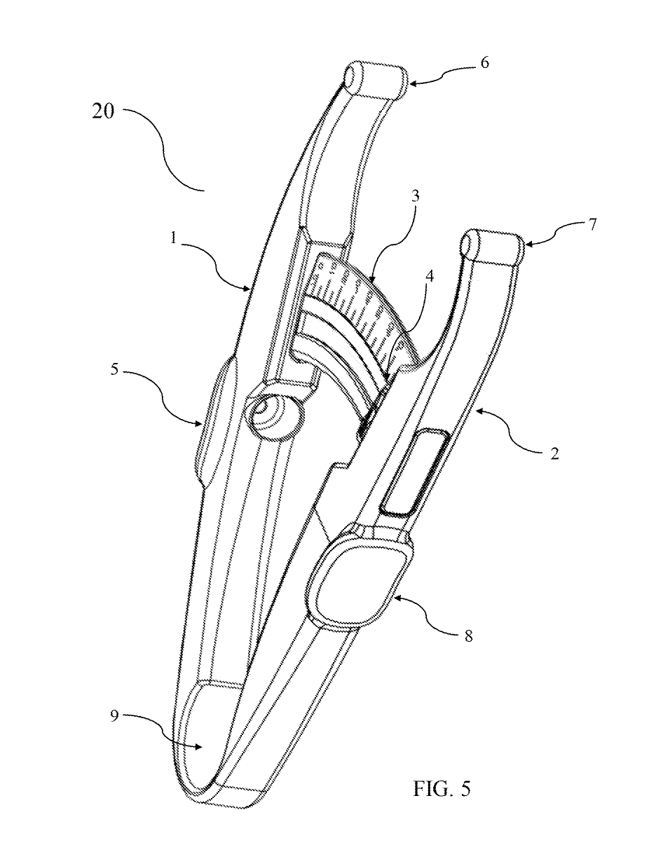

FIG. 5 is a vertical view of the skinfold caliper of the present invention more clearly showing indentation 8 for the middle finger, index finger, or the ring finger, to exert pressure on the second arm 2 when taking the skinfold thickness measurement.

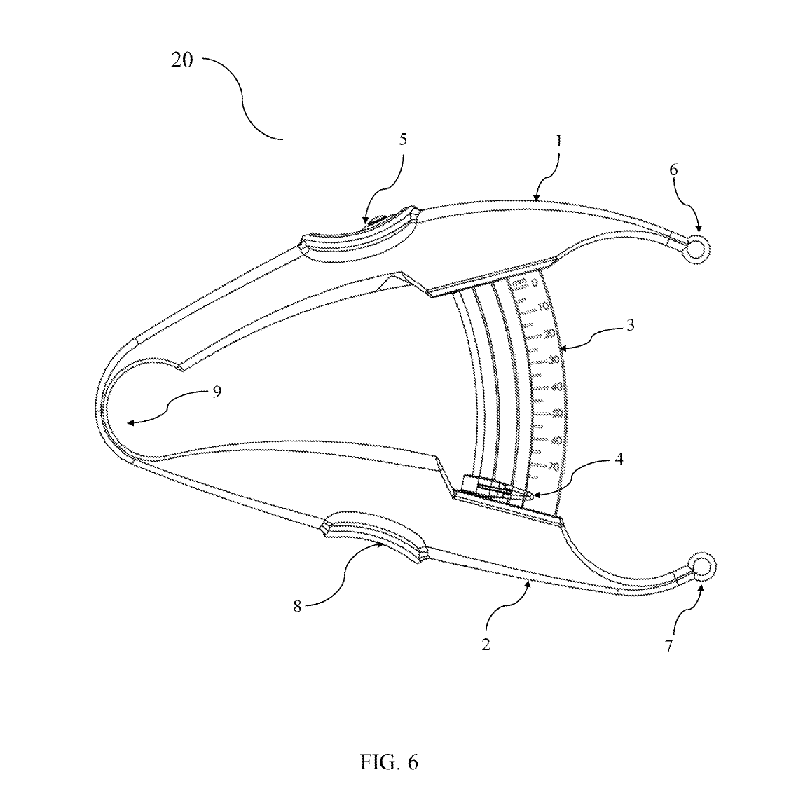

FIG. 6 is a side view of the skinfold caliper of the present invention illustrating the various exemplary parts of the device as described in the previous figures.

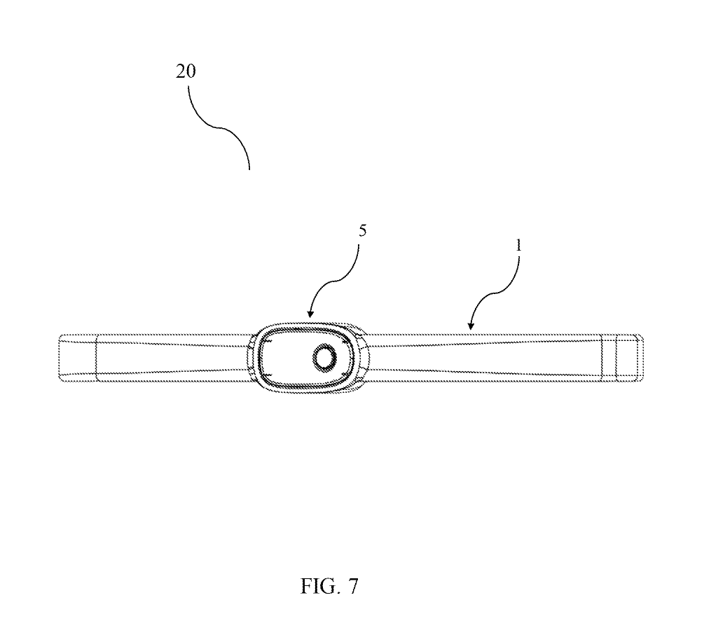

FIG. 7 is a top elevation view of the skinfold caliper of the present invention showing the first arm 1 with the button 5 used for depressing the arm When taking skinfold thickness measurements.

While the exemplary embodiment of the skinfold caliper of the present invention has thus been described through its preferred embodiment and related figures, it is to be understood that the embodiment of the present invention as described herein does not limit any application or scope of the invention and that the invention can be carried out and practiced in various ways and implemented in embodiments other than the embodiment outlined and described above. It should be understood and obvious to one skilled in the art that alternatives, modifications, and variations of the embodiment of the present invention as described may be construed as being within the spirit and scope of the appended claims.

What is claimed is:

1. A skinfold caliper comprising:
a first arm and a second arm;
said first arm having a skinfold contact structure means at a distal end;
said second arm having a skinfold contact structure means at a distal end;
said first arm and said second arm pivotally connected at a proximate end to provide a spring action of fully opening and closing of said first arm and said second arm;
said first arm having a means to place a thumb to depress said first arm to engage said structure means at said distal end of said first arm on a skinfold;
said means to place said thumb further comprising a button having an integrated pressure sensor;
said second arm having an indentation means to place a finger to depress said second arm to engage said structure means at said distal end of said second arm on a skinfold;
means to take a measurement of a thickness of said skinfold when said structure means at said distal end of said first arm and said structure means at said distal end of said second arm simultaneously engage a base of said skinfold;
said means to take said measurement of said thickness of said skinfold comprising a measurement ruler member having a measurement scale on a first side and a measurement scale on a second side of said measurement ruler member; and
a marker member to mark a measurement of a skinfold thickness on said measurement ruler.

2. The skinfold caliper of claim I wherein when the first arm of the caliper is pushed down by depressing the button on the first arm of the caliper with a thumb, and the second arm of the caliper is pushed up through the indentation means with a middle finger, an index finger, or a ring finger of the same hand, the contact structure means at the distal ends of the first arm and the second arm of the caliper come together to grasp a base of a skinfold held by an opposing hand to provide a measurement of the thickness of said skinfold, with said measurement being displayed on the measurement ruler member through the position of the marker member on the measurement ruler member.

3. The skinfold caliper of claim 1 wherein the contact structure means located at the distal ends of the first arm and the second arm are round-shaped to minimize a discomfort on a skinfold when the two distal ends of said first arm and said second arm pinch a base of a skinfold when taking a skinfold measurement.

4. The skinfold caliper of claim 1 wherein the integrated pressure sensor is located under the button on the first arm and senses a pressure applied to a skinfold when the first arm and the second arm are closed over a skinfold with the contact structure means at the distal ends of the first arm and said second arm grasping a base of said skinfold.

5. The skinfold caliper of claim 1 wherein said button has an embedded raised dome to provide a tactile feel and produces an audible click sound to signal to a user when an optimal skinfold compression is achieved.

6. The skinfold caliper of claim 1 wherein the button has an integrated leaf spring incorporated into an underside of the button to return the button to an original position when a pressure is released from the first aim of the caliper.

7. The skinfold caliper of claim 1 wherein said measurement ruler has a metric scale with measurements in millimeters on both sides of the ruler to enable reading a measurement from either side of the ruler by a right handed and a left handed person.

8. The skinfold caliper of claim 1 wherein the marker member is double-sided and detachable and re-attachable to the ruler.
